(19) United States
(12) Patent Application Publication
(10) Pub. No.: US 2010/0139294 A1
(43) Pub. Date: Jun. 10, 2010

(54) Cooling System Having A Bypass Valve To Regulate Fluid Flow

(75) Inventors: Mark Lowe, Danville, CA (US); Krister Bowman, Oakland, CA (US)

Correspondence Address: SHAY GLENN LLP, 2755 CAMPUS DRIVE, SUITE 210, SAN MATEO, CA, 94403, US

(73) Assignee: CoolSystems, Inc.

(21) Appl. No.: 12/329,461

(22) Filed: Dec. 5, 2008

(51) Int. Cl. F25D 3/00; F25B 41/04; F25B 49/00; G05D 23/00; A61F 7/00

(52) U.S. Cl. 62/56; 62/259.3; 62/196.1; 62/126; 62/157; 607/108

(57) ABSTRACT

A fluid chiller system supplying cold fluid to a therapeutic wrap, in which a minimum level of fluid flow is provided through the heat exchange coils to insure that the fluid in the coils does not freeze. A fluid flow meter or pressure sensor connected inline with the inlet port of the chiller unit measures a level of fluid flow. A controller is connected to the flow meter or pressure sensor and a bypass valve, wherein based on a signal from the flow meter or pressure sensor, the controller adjusts the bypass valve to provide a predetermined level of fluid flow into the inlet port of the chiller unit, in order to prevent the heat exchange coils from freezing, if the flow through the wrap is blocked.

### BACKGROUND OF THE INVENTION

1. Field of the Invention

The present invention relates generally to cooling systems, and more particularly, to a fluid chiller system supplying cold fluid to a therapeutic wrap, in which a minimum level of fluid flow is provided through the heat exchange coils to insure that the fluid in the coils does not freeze.

2. Description of the Related Art

Systems are known which provide active cooling and/or compression for humans and other animal bodies. They are used, for example, in physical therapy, pre-game conditioning, minor injury care, post-operative care, etc. In general, the body heat exchanging component(s) of such an apparatus has a pair of layers defining a flexible bladder through which a liquid is circulated. This component is often referred to as a “wrap.” The liquid circulated through the wrap is maintained at a desired temperature. Generally, the desired temperature is lower than the temperature expected for the body part, and typically is achieved, at least in part, by passing the liquid through a heat exchanging medium, such as by passing the same through an ice bath, or a refrigeration unit. One such system is disclosed, for example, in U.S. Pat. No. 6,178,562, the disclosure of which is herein incorporated by reference.

For certain applications, such as equine treatment, it is desirable to provide a temperature to the body part that is at or near the freezing point of the fluid circulated in the system. However, if the fluid circulating in the system for some reason has reduced flow, the fluid in the heat exchange unit may freeze in the coils, and cause the system to stop working.

U.S. Pat. No. 6,823,682 discloses an absorption chiller having a protection system for preventing chilled water in an evaporator from freezing in the event that the water flow through the system tubes stops while the machine is running. A sensor in the evaporator heat exchanger detects when the water flow through the heat exchanger tubes closes down, and signals a controller, and in turn shuts down the machine and opens a valve to deliver a high-temperature working fluid in order to maintain the temperature in the evaporator at a temperature above that at which the water in the tubes freeze.

The system described in U.S. Pat. No. 6,823,682 thus requires that the system be shut down when a low flow condition is detected, and that there be a high temperature fluid available for use.

European Patent Application No. EP 1 329 676 discloses a water refrigeration unit having a device to prevent the freezing of cooling fluid inside an evaporator. Based on the pressure in the system, the fluid in the system may be mixed with steam to raise the temperature of the fluid. However, such a system requires the availability of steam within the system.

U.S. Pat. No. 5,076,068 discloses a cooling device having a bypass valve downstream of a compressor. If a certain minimum pressure in the refrigeration circuit is not maintained in order to prevent the coolant in the system from freezing, a bypass valve is opened. When the pressure is above the minimum level, the bypass valve is closed. In other words, the bypass valve is either open or closed.

### SUMMARY OF THE INVENTION

In general, the present invention is a fluid chiller system supplying cold fluid to a therapeutic wrap, in which a minimum level of fluid flow is provided through the heat exchange coils to insure that the fluid in the coils does not freeze.

In one embodiment, a cooling system comprises a chiller unit having in outlet port and an inlet port, the chiller unit providing chilled fluid from the outlet port, a therapeutic wrap having in inlet port connected to the outlet port of the chiller unit, and an outlet port connected to the inlet port of the cooling unit, a bypass valve connected across the outlet and inlet ports of the chiller unit and the inlet and outlet lo ports of the therapeutic wrap, a fluid flow meter connected inline with the inlet port of the chiller unit, and a controller connected to the flow meter and bypass valve, wherein the controller receives a signal from the flow meter and adjusts the bypass valve to provide a predetermined level of fluid flow into the inlet port of the chiller unit.

The cooling unit may further comprise a refrigeration unit having heat exchange coils, a reservoir connected to the refrigeration unit to store fluid chilled by the refrigeration unit, and a pump to circulate the chilled fluid from the reservoir through the therapeutic wrap. The bypass valve may comprise a continuously variable valve, and the flow meter may comprise a positive displacement flow meter.

In one embodiment, when a system starts up, the controller opens the bypass valve for a predetermined period of time, or until the chilled fluid in the chiller unit reaches a predetermined temperature.

In an alternate embodiment, a pressure sensor can be used instead of a flow meter.

A method according to the present invention for preventing re-circulating fluid in a cooling system from freezing in heat exchange coils in a chiller unit comprises connecting a bypass valve across inlet and outlet ports of the chiller unit, detecting an amount of fluid flow into the heat exchange coils of the chiller unit, and adjusting the bypass valve to maintain a predetermined minimum level of fluid flow into the heat exchange coils, based on the detected amount of fluid flow.

### BRIEF DESCRIPTION OF THE DRAWINGS

The present invention will be readily understood by the following detailed description in conjunction with the accompanying drawings, wherein like reference numerals designate like structural elements, and in which:

FIG. 1 is a diagram of an embodiment of a system according to the present invention; and

FIG. 2 is a flowchart of an embodiment of a control sequence according to an aspect of the present invention.

### DETAILED DESCRIPTION OF THE INVENTION

The following description is provided to enable any person skilled in the art to make and use the invention and sets forth the best modes contemplated by the inventor for carrying out the invention. Various modifications, however, will remain readily apparent to those skilled in the art. Any and all such modifications, equivalents and alternatives are intended to fall within the spirit and scope of the present invention.

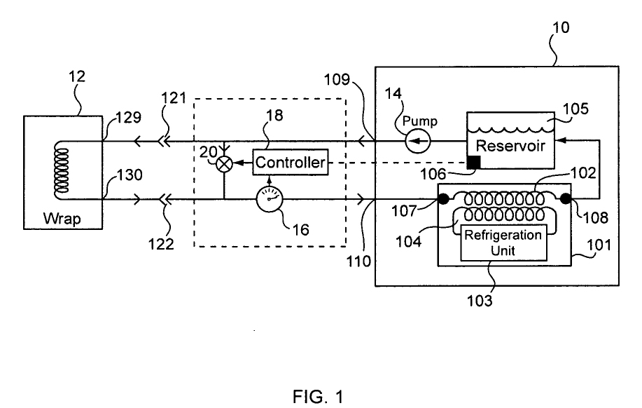

In general, the present invention is a fluid chiller system supplying cold fluid to a therapeutic wrap, in which a minimum level of fluid flow is provided through the heat exchange coils to insure that the fluid in the coils does not freeze. As shown in FIG. 1, an embodiment of the present invention includes a chiller unit 10 and a therapeutic wrap 12. The chiller unit 10 includes a cooling unit 101 having heat exchange coils 102 to cool the circulation fluid by means of, for example, a standard refrigeration unit 103 and evaporator coils 104. The cooled circulation is then stored in a reservoir 105. A pump 14 circulates fluid from the reservoir 105 through the wrap 12 and cooling unit 101.

Specifically, the pump 14 draws the chilled fluid out of the reservoir 105, and into the fluid circulation path via an outlet port 109. The fluid circulation path may comprise standard fluid tubing as is known in the art. The fluid flows into the inlet port 129 of the wrap 12, where the fluid is circulated through the wrap and is 1o generally warmed by the human or animal body heat. The fluid exits through an outlet port 130 and returns back to the chiller unit 10 via an inlet port 110.

The wrap 12 can be constructed, for example, as taught in U.S. Pat. Nos. 6,695,872 and 7,198,093, the disclosures of which is herein incorporated by reference. In addition, a control unit can provide air pressure to the wrap to provide compression, as taught by U.S. Pat. No. 6,695,872.

As noted above, if the fluid flow is reduced in the circulation path due to a kink in a hose, an obstruction in the wrap, etc. the fluid contained in the heat exchange coils 102 could freeze. Also, the wrap 12 can be connected to the chiller unit 10 via connectors 121 and 122. If a user inadvertently disconnects the wrap 12 from the chiller unit 10, the flow of fluid back into the cooling unit 101 would ordinarily stop. If as a result of the reduced fluid flow the fluid in the coils 102 freezes, the operation of the device would obviously be compromised, possibly requiring a user to stop the system and defrost the coils.

To overcome this problem, the present invention provides a flow meter 16 to measure the amount of fluid circulation in the circulation path. The flow meter 16 is preferably a positive displacement type flow meter. In one embodiment, by way of illustration and not limitation, a suitable flow meter is an Omega™ model FPD-1003, available from Omega Engineering, Inc. The flow meter 16 outputs a measurement value to a controller 18. Based on the flow rate through the flow meter 16, the controller 18 adjusts a bypass valve 20 to insure that a minimum amount of fluid is always circulating through the heat exchange coils 102. In a preferred embodiment, the controller 18 is a Cypress™ model CY8C29566 microcontroller, available from Cypress Semiconductor Corp. programmed to control the bypass valve 20, as described herein. The bypass valve 20 is preferably a continuously variable valve, such as a Burkert™ model 2835, with a 3 mm orifice, available from Burkert Fluid Control Systems.

For example, if a user inadvertently positions the wrap 12 such that the flow through the wrap is reduced, the flow meter 16 detects the reduced flow. The controller 18 will adjust the bypass valve 20 to allow fluid to flow directly from the outlet of the pump 14 back to the inlet of the heat exchange coils 102. This insures that there is sufficient fluid flow through the heat exchange coils 102 to prevent the fluid from freezing in the coils.

Once the blockage or obstruction is removed, the flow meter 16 detects the increased fluid flow and the controller 18 adjusts the bypass valve 20 to reduce the amount of fluid that bypasses the wrap 12.

In another embodiment, temperature sensors 107, 108 can be added to the inlet and outlet of the coils in the refrigeration unit to supply additional information to the controller. Specifically, the flow rate can be adjusted depending on the temperature of the fluid in the coils—the higher the temperature the less flow that is needed to prevent icing in the coils.

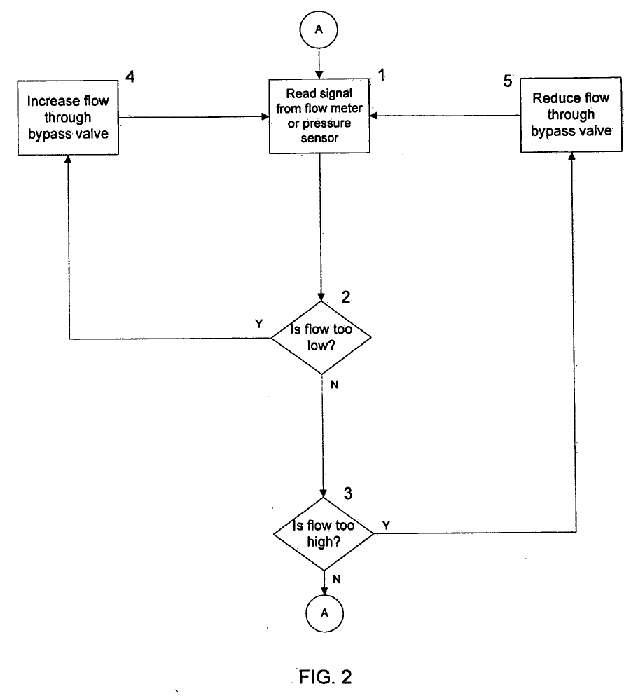

A flowchart of a control sequence according to one possible implementation is shown in FIG. 2. At step 1, the controller reads a signal from the flow meter or pressure sensor. If the flow level is too low at step 2, the controller adjusts the bypass valve to increase flow through the valve at step 4, and another reading is taken at step 1. If the flow rate is too high, the controller adjusts the bypass valve to reduce the flow level at step 5, and takes another reading at step 1. If the flow level is at a desired level or range, the process simply repeats, until there is a change in condition.

Note that the bypass valve can be adjusted in equal “steps” until a desired value (or range) is met. For faster control, the controller may adjust the bypass in larger increments at first, and then smaller increments to fine tune the flow level.

Note that in the preferred embodiment, the bypass valve 20 is a continuously variable valve. In other words, it is not necessarily only on or off, but can provide intermediate levels of flow to insure that the sum of the flow through the wrap, plus the flow through the bypass path is constant. This maximizes the flow through the wrap 12, and keeps the wrap 12 as cold as possible, regardless of the varying conditions in the fluid channels through the wrap 12. Unlike prior art systems which only have an on/off valve, the present invention can provide a more constant fluid flow through the heat exchange coils 102.

The present design has many other advantages over prior systems. For instance, the controller 18 and bypass valve 20 can be configured to maintain some pressure on the wrap 12, even when a blockage and/or kink is detected. Maintaining some pressure on the wrap can help encourage the blockage and/or kink to open up, so the system does not have to be stopped at the first sign of reduced flow.

In addition, the present invention allows for different viscosities of fluids to be used in the system, without requiring significant software updates to the controller. Also, unlike some prior art systems, the present invention does not require a separate heat source to provide heated fluid to the system to prevent the fluid in the coils from freezing, since a constant minimum flow is guaranteed.

Another advantage of the present design is that controller 20 can be programmed to provide maximum fluid bypass during system start-up. In other words, during a system start-up operation, the bypass valve 18 can be fully opened to allow a maximum amount of fluid to bypass the wrap 12. This allows the fluid in the circulation path to cool down to operating temperature as quickly as possible. After a predetermined period of time or after a predetermined fluid temperature in the reservoir 105 is reached (i.e. as determined by a temperature sensor 106 in the reservoir) the controller 20 can adjust the bypass valve 18 to its minimum position, thereby maximizing fluid flow through the wrap 12 for normal system operation.

While the present invention has been described using a flow meter to detect fluid flow through the circulation path, a pressure sensor could also be used in a similar manner in place of a fluid flow meter. In this alternate embodiment, as the detected pressure in the return line changes, the controller would adjust the bypass valve accordingly, to maintain a constant pressure in the fluid line i.e. a constant rate of fluid flow.

Those skilled in the art will appreciate that various adaptations and modifications of the just-described preferred embodiments can be configured without departing from the scope and spirit of the invention. Therefore, it is to be understood that, within the scope of the appended claims, the invention may be practiced other than as specifically described herein.

What is claimed is:

1. A cooling system comprising:
a chiller unit having an outlet port and an inlet port, the chiller unit providing chilled fluid from the outlet port;
a therapeutic wrap having in inlet port connected to the outlet port of the chiller unit, and an outlet port connected to the inlet port of the cooling unit;
a bypass valve connected across the outlet and inlet ports of the chiller unit and the inlet and outlet ports of the therapeutic wrap;
a fluid flow meter connected inline with the inlet port of the chiller unit; and
a controller connected to the flow meter and bypass valve;
wherein the controller receives a signal from the flow meter and adjusts the bypass valve to provide a predetermined level of fluid flow into the inlet port of the chiller unit.

2. The cooling system of claim 1, wherein the chiller unit comprises a refrigeration unit having heat exchange coils.

3. The cooling system of claim 2, wherein the chiller unit further comprises a reservoir connected to the refrigeration unit to store fluid chilled by the refrigeration unit.

4. The cooling system of claim 3, wherein the chiller unit further comprises a pump to circulate the chilled fluid from the reservoir through the therapeutic wrap.

5. The cooling system of claims 1, wherein the bypass valve comprises a continuously variable valve.

6. The cooling system of claim 5, wherein the flow meter is a positive displacement flow meter.

7. The cooling system of claim 1, wherein at a system start-up, the controller opens the bypass valve for a predetermined period of time.

8. The cooling system of claim 1, wherein at a system start-up, the controller opens the bypass valve until the chilled fluid in the chiller unit reaches a predetermined temperature.

9. The cooling system of claim 2, further comprising at least one temperature sensor in the refrigeration unit to determine a temperature of the fluid, wherein the controller adjusts the level of fluid flow based at least in part on a temperature level provided by the at least one temperature sensor.

10. A cooling system comprising:
a chiller unit having an outlet port and an inlet port, the chiller unit providing chilled fluid from the outlet port;
a therapeutic wrap having in inlet port connected to the outlet port of the chiller unit, and an outlet port connected to the inlet port of the chiller unit;
a bypass valve connected across the outlet and inlet ports of the chiller unit and the inlet and outlet ports of the therapeutic wrap;
a pressure sensor connected inline with the inlet port of the chiller unit; and
a controller connected to the flow meter and bypass valve;
wherein the controller receives a signal from the pressure sensor and adjusts the bypass valve to provide a predetermined level of fluid flow into the inlet port of the chiller unit.

11. The cooling system of claim 10, wherein the chiller unit comprises a refrigeration unit having heat exchange coils.

12. The cooling system of claim 11, wherein the chiller unit further comprises a reservoir connected to the refrigeration unit to store fluid chilled by the refrigeration unit.

13. The cooling system of claim 12, wherein the chiller unit further comprises a pump to circulate the chilled fluid from the reservoir through the therapeutic wrap.

14. The cooling system of claims 10, wherein the bypass valve comprises a continuously variable valve.

15. The cooling system of claim 10, wherein at a system start-up, the controller opens the bypass valve for a predetermined period of time.

16. The cooling system of claim 10, wherein at a system start-up, the controller opens the bypass valve until the chilled fluid in the chiller unit reaches a predetermined temperature.

17. The cooling system of claim 10, further comprising at least one temperature sensor in the refrigeration unit to determine a temperature of the fluid, wherein the controller adjusts the level of fluid flow based at least in part on a temperature level provided by the at least one temperature sensor.

18. A method for preventing re-circulating fluid in a cooling system from freezing in heat exchange coils in a chiller unit, the method comprising:
connecting a bypass valve across inlet and outlet ports of the chiller unit;
detecting an amount of fluid flow into the heat exchange coils of the chiller unit; and
adjusting the bypass valve to maintain a predetermined minimum level of fluid flow into the heat exchange coils, based on the detected amount of fluid flow.

19. The method of claim 18, further comprising determining a temperature of the fluid in the heat exchange coils, and adjusting the bypass valve based at least in part on a temperature level provided by the determined temperature.
